(19) United States
(12) Patent Application Publication
(10) Pub. No.: US 2026/0179175 A1
(43) Pub. Date: Jun. 25, 2026

(54) SYSTEM AND METHOD FOR GENERATING CUSTOMIZED SCHEMATIC VIEWS OF CORONARY ARTERIES

(71) Applicant: GE Precision Healthcare LLC, Waukesha, WI (US)

(72) Inventors: Jorge Eduardo Hernandez Londono, Versailles (FR); Romane Amice, Paris (FR); Sherazade Aknoun, Verrières Le Buisson (FR); Racha Hachem, Massy (FR); Paul Jules Guillemot, Séné (FR)

(21) Appl. No.: 18/988,312

(22) Filed: Dec. 19, 2024

(51) Int. Cl. G06T 3/067; A61B 6/03; G06T 17/00

(52) U.S. Cl. CPC G06T 3/067; A61B 6/032; G06T 17/005

(57) ABSTRACT

A computer-implemented method includes obtaining, via a processing system comprising one or more processors, a three-dimensional (3D) coronary tree segmented from computed tomography (CT) cardiac scan data of a subject, wherein the 3D coronary tree is labeled. The computer-implemented method also includes obtaining, via the processing system, a standardized two-dimensional (2D) coronary base schematic representation of a generic coronary tree. The computer-implemented method further includes modifying, via the processing system, the standardized 2D coronary base schematic representation of the generic coronary tree based on the 3D coronary tree to generate a standardized 2D coronary personalized schematic representation of a coronary tree of the subject.

### BACKGROUND

The subject matter disclosed herein relates to imaging systems and, more particularly, to a system and a method for generating customized schematic views of coronary arteries.

Volumetric medical imaging technologies use a variety of techniques to gather three-dimensional information about the body. For example, a computed tomography (CT) imaging system measures the attenuation of X-ray beams passed through a patient from numerous angles. Based upon these measurements, a computer is able to reconstruct cross-sectional images of the portions of a patient's body responsible for the radiation attenuation. As will be appreciated by those skilled in the art, these images are based upon separate examination of a series of angularly-displaced measurements. It should be pointed out that a CT system produces data that represent the distribution of linear attenuation coefficients of the scanned object. The data are then reconstructed to produce an image that is typically displayed on a screen and may be printed or reproduced on film.

For example, in the field of CT angiography (CTA), vasculature and other circulatory system structures may be imaged, typically by administration of a radio-opaque dye prior to imaging. Visualization of the CTA data typically is performed in a two-dimensional (2D) manner, i.e., slice-by-slice, or in a three-dimensional (3D) manner, i.e., volume visualization, which allows the data to be analyzed for vascular pathologies. For example, the data may be analyzed for aneurysms, vascular calcification, renal donor assessment, stent placement, vascular blockage, and vascular evaluation for sizing and/or runoff. Once a pathology is located, quantitative assessments of the pathology may be made.

Currently, vasculature tree representations may be provided in 3D or through 2D projections that may not be user-friendly. In the case of 3D presentation, the appearance of the vascular tree representation varies based on the viewing angle. In addition, some of these presentations may not be easily interpretable.

### SUMMARY

Certain embodiments commensurate in scope with the originally claimed subject matter are summarized below. These embodiments are not intended to limit the scope of the claimed subject matter, but rather these embodiments are intended only to provide a brief summary of possible forms of the subject matter. Indeed, the subject matter may encompass a variety of forms that may be similar to or different from the embodiments set forth below.

In one embodiment, a computer-implemented method for generating a customized schematic view of coronary arteries is provided. The computer-implemented method includes obtaining, via a processing system comprising one or more processors, a three-dimensional (3D) coronary tree segmented from computed tomography (CT) cardiac scan data of a subject, wherein the 3D coronary tree is labeled. The computer-implemented method also includes obtaining, via the processing system, a standardized two-dimensional (2D) coronary base schematic representation of a generic coronary tree. The computer-implemented method further includes modifying, via the processing system, the standardized 2D coronary base schematic representation of the generic coronary tree based on the 3D coronary tree to generate a standardized 2D coronary personalized schematic representation of a coronary tree of the subject.

In another embodiment, a system for generating a customized schematic view of coronary arteries is provided. The system includes a memory encoding processor-executable routines. The system also includes a processing system including one or more processors and configured to access the memory and to execute the processor-executable routines, wherein the processor-executable routines, when executed by the processing system, cause the processing system to perform actions. The actions include obtaining a three-dimensional (3D) coronary tree segmented from computed tomography (CT) cardiac scan data of a subject, wherein the 3D coronary tree is labeled. The actions also include obtaining a standardized two-dimensional (2D) coronary base schematic representation of a generic coronary tree. The actions further include modifying the standardized 2D coronary base schematic representation of the generic coronary tree based on the 3D coronary tree to generate a standardized 2D coronary personalized schematic representation of a coronary tree of the subject.

In a further embodiment, a non-transitory computer-readable medium, the computer-readable medium including processor-executable code that when executed by a processing system including one or more processors, causes the processing system to perform actions. The actions include obtaining a three-dimensional (3D) coronary tree segmented from computed tomography (CT) cardiac scan data of a subject, wherein the 3D coronary tree is labeled. The actions also include obtaining a standardized two-dimensional (2D) coronary base schematic representation of a generic coronary tree. The actions further include modifying the standardized 2D coronary base schematic representation of the generic coronary tree based on the 3D coronary tree to generate a standardized 2D coronary personalized schematic representation of a coronary tree of the subject.

### BRIEF DESCRIPTION OF THE DRAWINGS

These and other features, aspects, and advantages of the disclosed subject matter will become better understood when the following detailed description is read with reference to the accompanying drawings in which like characters represent like parts throughout the drawings, wherein:

FIG. 1 is a combined pictorial view and block diagram of a computed tomography (CT) imaging system as discussed herein;

FIG. 2 is a schematic diagram of a computing device for performing the disclosed techniques, in accordance with aspects of the present disclosure;

FIG. 3 is a schematic diagram of a process for generating customized schematic views of coronary arteries, in accordance with aspects of the present disclosure;

FIG. 4 is a flow chart of a method for identifying unknown branches in a 3D segmentation of a coronary tree of a subject, in accordance with aspects of the present disclosure;

FIG. 5 depicts an image of a 3D coronary tree segmentation during identification of unknow branches, in accordance with aspects of the present disclosure;

FIG. 6 is a schematic diagram illustrating generation of a standardized 2D coronary base schematic representation of a generic coronary tree, in accordance with aspects of the present disclosure;

FIG. 7 is a flow chart of a method for generating customized schematic views of coronary arteries, in accordance with aspects of the present disclosure;

FIG. 8 depicts a schematic diagram of a first step in manipulating a standardized 2D coronary base schematic representation of a generic coronary tree (i.e., identifying anomalies), in accordance with aspects of the present disclosure;

FIG. 9 depicts the rendering of different coronary base schematic representations after initial manipulation, in accordance with aspects of the present disclosure;

FIG. 10 depicts examples of rendering different base schematic representation after initial manipulation (e.g., with no left main), in accordance with aspects of the present disclosure;

FIG. 11 depicts a schematic diagram of a second step in manipulating a standardized 2D coronary base schematic representation of a generic coronary tree (i.e., removing non-existing branches), in accordance with aspects of the present disclosure;

FIG. 12 depicts a schematic diagram of a third step in manipulating a standardized 2D coronary base schematic representation of a generic coronary tree (i.e., changing length of sub-branches), in accordance with aspects of the present disclosure;

FIG. 13 depicts a schematic diagram of a fourth step in manipulating a standardized 2D coronary base schematic representation of a generic coronary tree (i.e., changing bifurcation point), in accordance with aspects of the present disclosure;

FIG. 14 depicts a schematic diagram of the generation of a standardized 2D coronary personalized representation of a coronary tree of a subject, in accordance with aspects of the present disclosure;

FIG. 15 depicts a user interface depicting a final rendering of a standardized 2D coronary personalized representation of a coronary tree of a subject, in accordance with aspects of the present disclosure; and

FIG. 16 depicts another user interface depicting a 3D coronary tree of a subject and a final rendering of a standardized 2D coronary personalized representation of a coronary tree of the subject derived from the 3D coronary tree, in accordance with aspects of the present disclosure.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers'specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present subject matter, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

Currently, vascular tree representations are frequently presented in 3D, with their appearance varying based on the viewing angle. This variability highlights the need for a standardized yet customizable schematic representation.

The present disclosure provides embodiments for a system and a method for generating customized schematic views of coronary arteries. The disclosed embodiments include the generation of simplified, personalized 2D diagram of segmented cardiovascular systems based on a standard (e.g., provided by the Society of Cardiovascular Computed Tomography (SCCT). In particular, a generalized or base 2D schematic representation of the coronary arteries is adapted to integrate personalized features from automatic 3D coronary segmentation and labeling to generate a personalized schematic representation of a coronary tree of the patient (e.g., subject). The personalized schematic representation accurately depicts the patient's unique vascular anatomy. The personalized schematic representation also references a universally understood standardized diagram. The personalized schematic representation further provides a complete view independent of viewing angle. The personalized schematic representation also complements existing 3D visualizations. The personalized schematic representation is also suitable for inclusion in clinical reports.

The disclosed embodiments bridge the gap between detailed 3D imagery and the need for standardized, easily interpretable 2D schematics in clinical settings based on a standard provided by SCCT. By providing a more accessible and comprehensive visualization tool, it aims to improve communication among healthcare professionals and enhance the overall quality of coronary artery disease assessment and treatment planning.

Although the following discusses the disclosed embodiments with regard to CT imaging systems, the techniques described herein may apply to other types of imaging systems. For example, the disclosed techniques may apply to an MRI system or a nuclear medicine imaging system such as a PET imaging system or a SPECT imaging system. The disclosed techniques may also apply to medical imaging systems having a combination of the above medical imaging modalities.

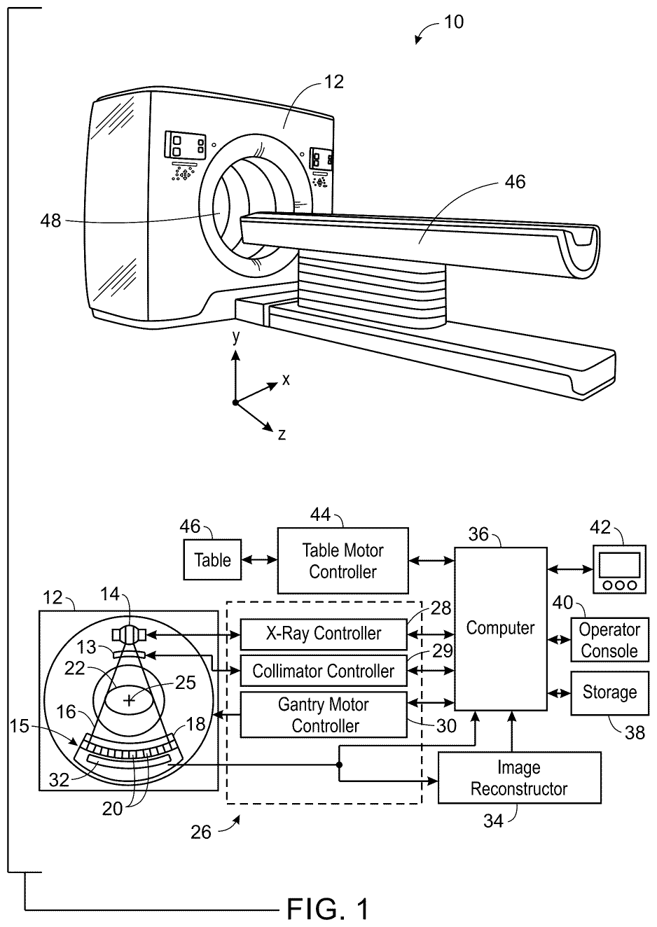

With the preceding in mind and referring to FIG. 1, a computed tomography (CT) imaging system 10 is shown, by way of example. The CT imaging system 10 includes a gantry 12. The gantry 12 has an X-ray source 14 that projects a beam of X-rays 16 toward a detector assembly 15 on the opposite side of the gantry 12. The X-ray source 14 projects the beam of X-rays 16 through a pre-patient collimator assembly 13 that determines the size and shape of the beam of X-rays 16. The detector assembly 15 includes a collimator assembly 18 (a post-patient collimator assembly), a plurality of detector modules 20 (e.g., detector elements or sensors), and data acquisition systems (DAS) 32. The plurality of detector modules 20 detect the projected X-rays that pass through a subject or object 22 being imaged, and DAS 32 converts the data into digital signals for subsequent processing. Each detector module 20 in a conventional system produces an analog electrical signal that represents the intensity of an incident X-ray beam and hence the attenuated beam as it passes through the subject or object 22. During a scan to acquire X-ray projection data, gantry 12 and the components mounted thereon rotate about a center of rotation 25 (e.g., isocenter) so as to collect attenuation data from a plurality of view angles relative to the imaged volume.

Rotation of gantry 12 and the operation of X-ray source 14 are governed by a control system 26 of CT imaging system 10. Control system 26 includes an X-ray controller 28 that provides power and timing signals to an X-ray source 14, a collimator controller 29 that controls a length and a width of an aperture of the pre-patient collimator 13 (and, thus, the size and shape of the beam of X-rays 16), and a gantry motor controller 30 that controls the rotational speed and position of gantry 12. An image reconstructor 34 receives sampled and digitized X-ray data from DAS 32 and performs high-speed image reconstruction. The reconstructed image is applied as an input to a computer 36, which stores the image in a storage device 38. Computer 36 also receives commands and scanning parameters from an operator via console 40. An associated display 42 allows the operator to observe the reconstructed image and other data from computer 36. The operator supplied commands and parameters are used by computer 36 to provide control signals and information to DAS 32, X-ray controller 28, collimator controller 29, and gantry motor controller 30. In addition, computer 36 operates a table motor controller 44, which controls a motorized table 46 to position subject 22 and gantry 12. Particularly, table 46 moves portions of subject 22 through a gantry opening or bore 48.

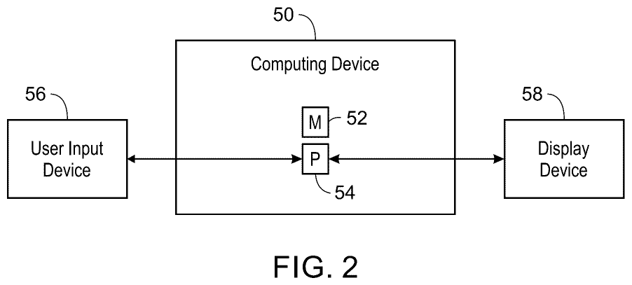

FIG. 2 is a schematic diagram of a computing device 50 for performing the disclosed techniques herein. The computing device 50 may be computer 36 of the CT imaging system 10 in FIG. 1 or a remote computing device. In certain embodiments, the computing device 50 may be a remote cloud-based processing system.

The computing device 50 includes a memory 52 and a processing system 54. In some embodiments, the processing system 54 may include one or more general purpose processors, one or more application specific integrated circuits, one or more field programmable gate arrays, or the like. Additionally, the memory 52 may be any tangible, non-transitory, computer readable medium that is capable of storing instructions executable by the processing system 54 and/or data that may be processed by the processor 54. In other words, the memory 52 may include volatile memory, such as random-access memory, or non-volatile memory, such as hard disk drives, read only memory, optical disks, flash memory, and the like. The memory 52 may store imaging data, patient-related data, neural network framework for analysis of detecting localizing coronary artery calcifications, personalized schematic generation software, and other data.

The computing device 50 is communicatively coupled with a user input device 56 and a display device 58. The user input device 56 may include one or more of a touchscreen, a keyboard, a mouse, a trackpad, a motion sensing camera, or other device configured to enable a user to interact with the computing device 50. The display device 58 may include one or more display devices utilizing virtually any type of technology. In some embodiments, the display device 58 may include a computer monitor, and may display imaging data (e.g., CT cardiac imaging data), 3D coronary tree, and a personalized schematic representation of a coronary tree of a patient (and associated information). The display device 58 may be combined with the processing system 54, the non-transitory memory 52, and/or the user input device 56 in a shared enclosure, or may be peripheral display devices and may comprise a monitor, touchscreen, projector, or other display device known in the art, which may enable a user to view data and/or interact with various data stored in the non-transitory memory 52.

As described in greater detail below, the processing system 54 is configured to obtain (e.g., receive or access) vascular or cardiac imaging data (e.g., CT angiography (CTA) data or CT cardiac scan data) from a subject (e.g., patient). In certain embodiments (e.g., when the computing device 50 is part of the CT imaging system 10), the processing system 54 is configured (via the CT imaging system 10) to acquire the vascular or cardiac imaging data. In certain embodiments, the processing system 54 is configured to segment the vascular or cardiac imaging data to generate 3D coronary tree from the vascular or cardiac imaging data. In certain embodiments, the processing system 54 is configured to label the 3D coronary tree. In certain embodiments, the processing system 54 may utilize a trained deep learning-based model or network for the segmentation and labeling (e.g., CardIQ Suite from GE Healthcare). The trained deep learning-based model or network maybe configured to also rapidly detect and localize coronary artery calcifications and to generate comprehensive calcification scores for the entire coronary system as well as individual arterial territories. In certain embodiments, the processing system 54 is configured to obtain the 3D coronary tree of the subject (e.g., already segmented and labeled).

The processing system 54 is configured to obtain a standardized 2D coronary base schematic representation of a generic coronary tree. The standardized 2D coronary base schematic representation of the generic coronary tree is standardized based on standardized coronary segmentation tree diagram provided by SCCT. In certain embodiments, the standardized 2D coronary base schematic representation of the generic coronary tree is derived from a combination of a right-dominant SCCT coronary tree segmentation diagram, a co-dominant SCCT coronary segmentation tree diagram, and a left-dominant SCCT coronary tree segmentation diagram.

The processing system 54 is also configured to modify the standardized 2D coronary base schematic representation of the generic coronary tree based on the 3D coronary tree to generate a standardized 2D coronary personalized schematic representation of a coronary tree of the subject. Since the standardized 2D coronary base schematic representation of the generic coronary tree is standardized, the generated standardized 2D coronary personalized schematic representation of the coronary tree of the subject is also standardized (with respect to SCCT). In certain embodiments, modifying the standardized 2D base schematic representation of the generic coronary tree based on the 3D coronary tree includes: identifying anomalies in the 3D coronary tree; removing any branch from the standardized 2D coronary base schematic representation of the generic coronary tree that does not exist in the 3D coronary tree; changing a respective length of one or more sub-branches of the standardized 2D coronary base schematic representation of the generic coronary tree based on a corresponding respective length of the one or more corresponding sub-branches in the 3D coronary tree; and changing a bifurcation point of the standardized 2D coronary base schematic representation of the generic coronary tree based on a corresponding bifurcation point of the 3D coronary tree.

In certain embodiments, prior to modifying the standardized 2D coronary base schematic representation of the generic coronary tree, the processing system 54 is configured to identify unknown branches in the 3D coronary tree. Identifying unknown branches in the 3D coronary tree may include: defining a list of unknown branches with their coordinates; defining a list of main branches with their coordinates; defining a list of possible sub-branches for the main branches from the list of unknown branches; identifying existing sub-branches of each main branch; identifying common parts between a respective unknown branch from the list of possible sub-branches and a respective main branch; determining that a respective common part is a sub-branch when the respective common part is greater than an aorta of the 3D coronary tree; and adding the determined sub-branch to the identified existing sub-branches.

The processing system 54 is also configured to display the standardized 2D coronary personalized schematic representation of the coronary tree of the subject on a graphical user interface (on the display device 58). The appearance of the standardized 2D coronary personalized schematic representation of the coronary tree of the subject on the graphical user interface is independent of viewing angle.

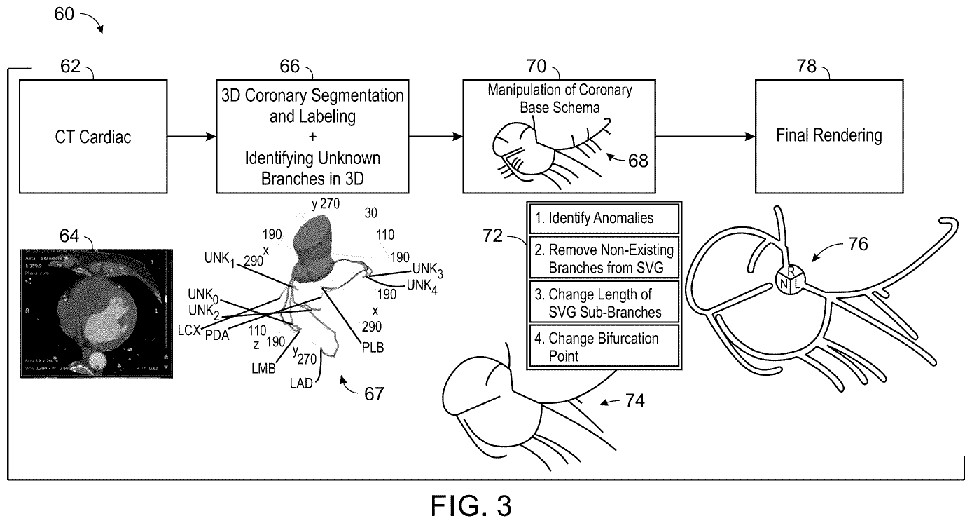

FIG. 3 is a schematic diagram of a process 60 for generating customized schematic views of coronary arteries. As depicted, the process 60 includes obtaining CT cardiac scan data of a subject (e.g., patient) as indicated by reference numeral 62. Image 64 depicts a CT image of the cardiac region of a subject. The process 60 also includes performing 3D coronary segmentation and labeling on the CT cardiac scan data of the subject as indicated by reference numeral 66. The process 60 further includes identifying unknown branches in the 3D coronary segmentation also as indicated by reference numeral 66. In certain embodiments, a trained deep learning-based model or network for the segmentation and labeling (e.g., CardIQ Suite from GE Healthcare). The trained deep learning-based model or network maybe configured to also rapidly detect and localize coronary artery calcifications and to generate comprehensive calcification scores for the entire coronary system as well as individual arterial territories. Image 67 depicts the 3D coronary tree segmented the CT cardiac scan data of the subject and labeled. As described in greater detail below, identifying unknown branches in the 3D coronary tree may include: defining a list of unknown branches with their coordinates; defining a list of main branches with their coordinates; defining a list of possible sub-branches for the main branches from the list of unknown branches; identifying existing sub-branches of each main branch; identifying common parts between a respective unknown branch from the list of possible sub-branches and a respective main branch; determining that a respective common part is a sub-branch when the respective common part is greater than an aorta of the 3D coronary tree; and adding the determined sub-branch to the identified existing sub-branches.

The process 60 further includes manipulating or modifying a coronary base (generic) schema 68 that is standardized to the SCCT based on the labeled 3D coronary tree of the subject as indicated by reference numeral 70. As depicted by reference numeral 72, manipulating or modifying the standardized 2D base schematic representation of the generic coronary tree (i.e., the scalable vector graphic (SVG) of the standardized 2D base schematic representation ) based on the 3D coronary tree includes: identifying anomalies in the 3D coronary tree; removing any branch from the standardized 2D coronary base schematic representation of the generic coronary tree (i.e., SVG) that does not exist in the 3D coronary tree; changing a respective length of one or more sub-branches of the standardized 2D coronary base schematic representation (i.e., SVG) of the generic coronary tree based on a corresponding respective length of the one or more corresponding sub-branches in the 3D coronary tree; and changing a bifurcation point of the standardized 2D coronary base schematic representation of the generic coronary tree based on a corresponding bifurcation point of the 3D coronary tree. This manipulation or modification generates an initial standardized 2D coronary personalized schematic representation of the coronary tree of the subject indicated by reference numeral 74. The process 60 even further includes providing and displaying a finalized rendering 76 of the standardized 2D coronary personalized schematic representation of the coronary tree of the subject as indicated by reference numeral 78.

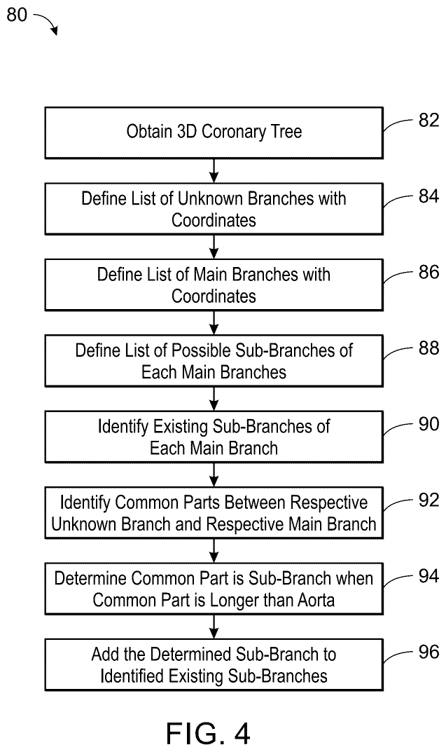

FIG. 4 is a flow chart of a method 80 for identifying unknown branches in a 3D segmentation of a coronary tree of a subject (e.g., patient). Some or all of the steps of the method 80 may be performed by the computing device 50 in FIG. 2.

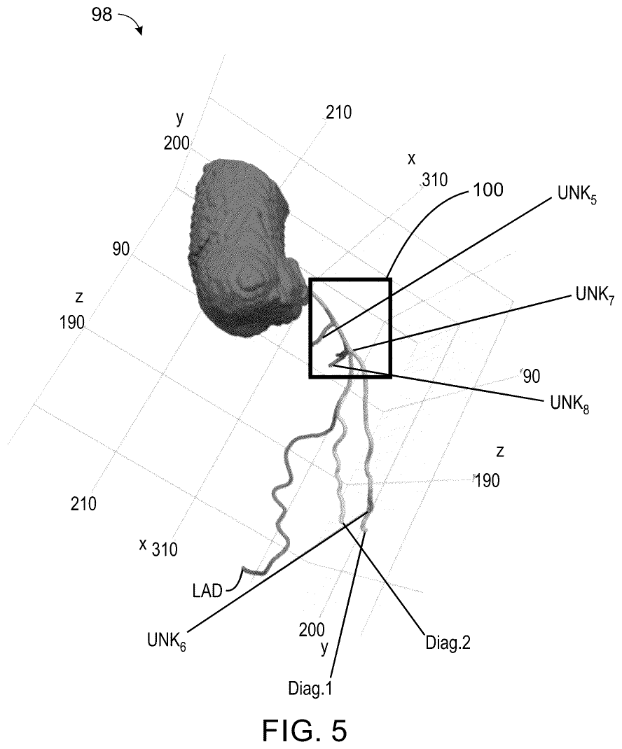

The method 80 includes obtaining a 3D coronary tree segmented CT cardiac scan data of a subject (block 82). The method 80 also includes defining a list of unknown (UNK) branches with their coordinates (block 84). The method 80 further includes defining a list of main branches (right coronary artery (RCA), left circumflex (LCX) artery, and left anterior descending (LAD) artery) with their coordinates (block 86). The method 80 further includes defining a list of possible sub-branches for the main branches from the list of unknown branches (block 88). The method 80 even further includes identifying existing sub-branches of each main branch (block 90). The method 80 still further includes identifying common parts between a respective unknown branch from the list of possible sub-branches and a respective main branch (block 92). The method 80 yet further includes determining that a respective common part is a sub-branch when the respective common part is greater than an aorta of the 3D coronary tree (block 94). The method 80 further includes adding the determined sub-branch to the identified existing sub-branches (block 96). Image 98 in FIG. 5 depicts an example of 3D coronary tree 100 segmented from CT cardiac scan data of a subject. Rectangle 100 in the image 98 highlights unknown branches.

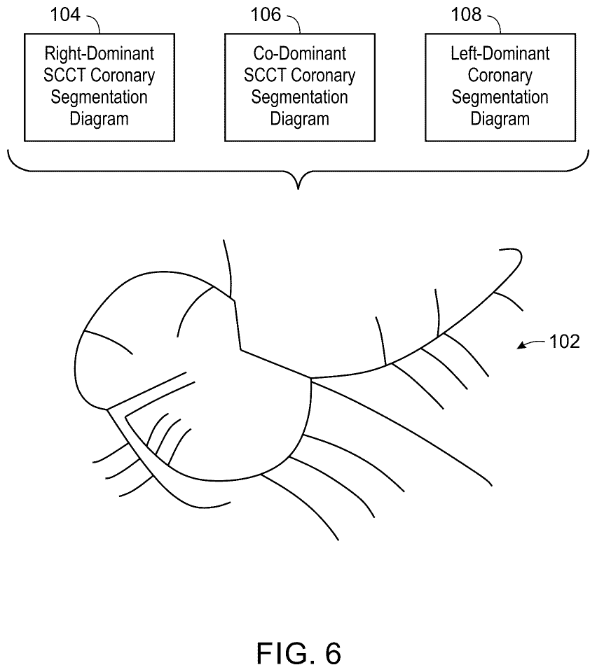

FIG. 6 is a schematic diagram illustrating generation of a standardized 2D coronary base schematic representation 102 of a generic coronary tree. The standardized 2D coronary base schematic representation 102 is standardized based on standardized coronary segmentation tree diagram provided by SCCT. In particular,, the standardized 2D coronary base schematic representation 102 of the generic coronary tree is derived from a combination of a right-dominant SCCT coronary tree segmentation diagram 104, a co-dominant SCCT coronary segmentation tree diagram 106, and a left-dominant SCCT coronary tree segmentation diagram 108 as depicted in FIG. 6.

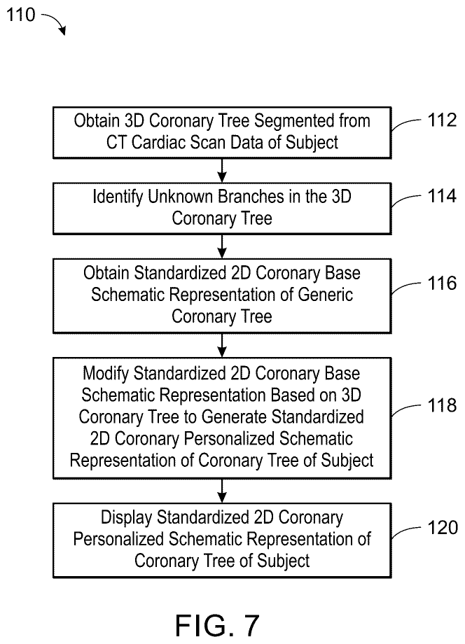

FIG. 7 is a flow chart of a method 110 for generating customized schematic views of coronary arteries. Some or all of the steps of the method 110 may be performed by the computing device 50 in FIG. 2.

The method 110 includes obtaining a 3D coronary tree segmented from CT cardiac scan data of a subject, wherein the 3D coronary tree is labeled (block 112). The method 110 includes identifying unknown branches in the 3D coronary tree as described in the method 80 in FIG. 4 (block 114). The method 110 also includes obtaining a standardized 2D coronary base schematic representation of a generic coronary tree (block 116). The method 110 further includes modifying the standardized 2D coronary base schematic representation of the generic coronary tree based on the 3D coronary tree to generate a standardized 2D coronary personalized schematic representation of a coronary tree of the subject (block 118). Modifying the standardized 2D coronary base schematic representation of the generic coronary tree (which is described in greater detail below) includes identifying anomalies in the 3D coronary tree; removing any branch from the standardized 2D coronary base schematic representation of the generic coronary tree (i.e., SVG) that does not exist in the 3D coronary tree; changing a respective length of one or more sub-branches of the standardized 2D coronary base schematic representation (i.e., SVG) of the generic coronary tree based on a corresponding respective length of the one or more corresponding sub-branches in the 3D coronary tree; and changing a bifurcation point of the standardized 2D coronary base schematic representation of the generic coronary tree based on a corresponding bifurcation point of the 3D coronary tree. The method 110 even further includes displaying the standardized 2D coronary personalized schematic representation of the coronary tree of the subject on a graphical user interface (block 120). The appearance of the standardized 2D coronary personalized schematic representation of the coronary tree of the subject on the graphical user interface is independent of viewing angle.

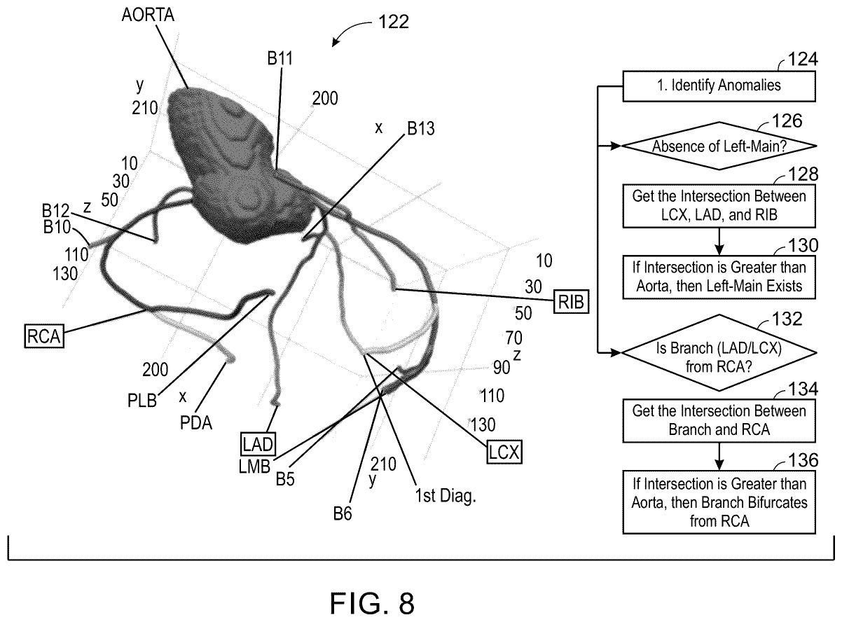

FIGS. 8 and 11-14 depict the process of manipulating a standardized 2D coronary base schematic representation of a generic coronary tree. After each step of the manipulation of the standardized 2D coronary base schematic representation of a generic coronary tree, the standardized 2D coronary base schematic representation may be also considered an intermediate 2D coronary personalized schematic representation of the coronary tree of the subject until generating and then rendering the final 2D coronary personalized schematic representation of the coronary tree of the subject.

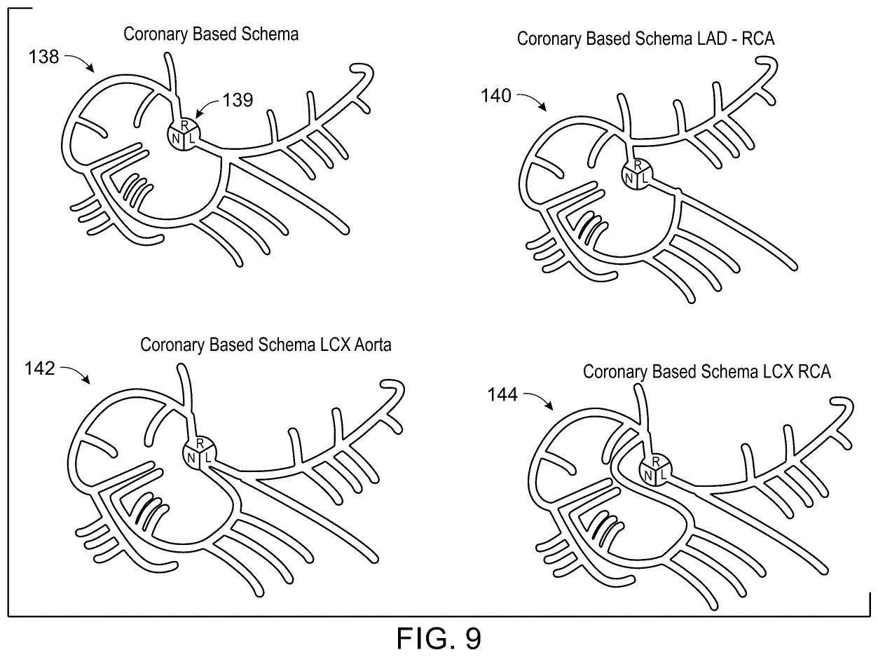

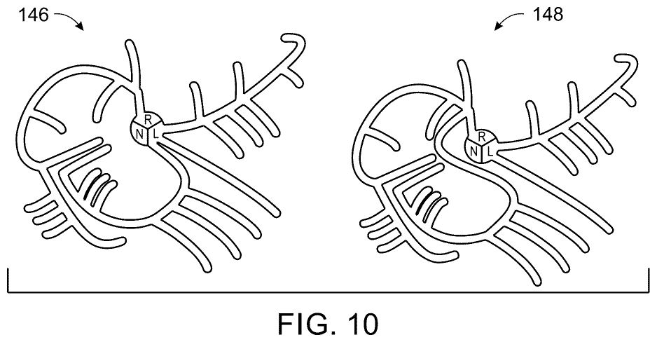

FIG. 8 depicts a schematic diagram of a first step in manipulating a standardized 2D coronary base schematic representation of a generic coronary tree. The left side of FIG. 8 depicts an image 122 of a 3D coronary tree of a subject (derived from cardiac CT scan data) utilized to manipulate a standardized 2D coronary base schematic representation of a generic coronary tree. The first step includes identifying any anomalies in the 3D coronary tree of the subject as indicated by reference numeral 124. For example, a determination is made of whether a left-main is absent as indicated by reference numeral 126. This determination is made by getting the intersection between LCX, LAD, and ramus intermediate branch (RIB) as indicated by reference numeral 128. If the intersection is greater than the aorta, then the left-main exists as indicated by reference numeral 130. Also, a determination is made if a respective branch (LAD/LCX) is from the RCA as indicated by reference numeral 134. This determination is made by getting the intersection between the respective branch and the RCA as indicated by indicated by reference numeral 134. If the intersection is greater than the aorta, then the respective branch bifurcates from RCA as indicated by reference numeral 136. In certain embodiments, after identifying any anomalies in the 3D coronary tree, the standardized 2D coronary base schematic representation of a generic coronary tree (i.e., coronary base schema) (e.g., indicated by reference numeral 138 in FIG. 9) may be directly initially manipulated. In certain embodiments, after identifying any anomalies in the 3D coronary tree (i.e., identifying the main topology), a standardized 2D coronary base schematic representation of a generic coronary tree may be chosen from a plurality of different standardized 2D coronary base schematic representations of a generic coronary tree with different main topologies shown in FIGS. 9 and 10.

FIG. 9 depicts the rendering of different coronary base schematic representations after initial manipulation (e.g., after identifying any anomalies). The upper left-hand corner of FIG. 9 depicts a coronary base schema 138 before manipulation. Region 139 represents the origin of the coronary arteries. N represents non-coronary sinus, R represents right sinus, and L represents left sinus. The upper right-hand corner of FIG. 9 depicts a coronary base schema LAD-RCA after initial manipulation of the coronary base schema 140. The lower left-hand corner of FIG. 9 depicts a coronary base schema LCX aorta 142. The lower right-hand corner of FIG. 9 depicts a coronary base schema LCX RCA 144. FIG. 10 depicts additional examples of rendering different base schematic representation after initial manipulation. The coronary base schema 146 and the coronary base schema 148 both lack a left main.

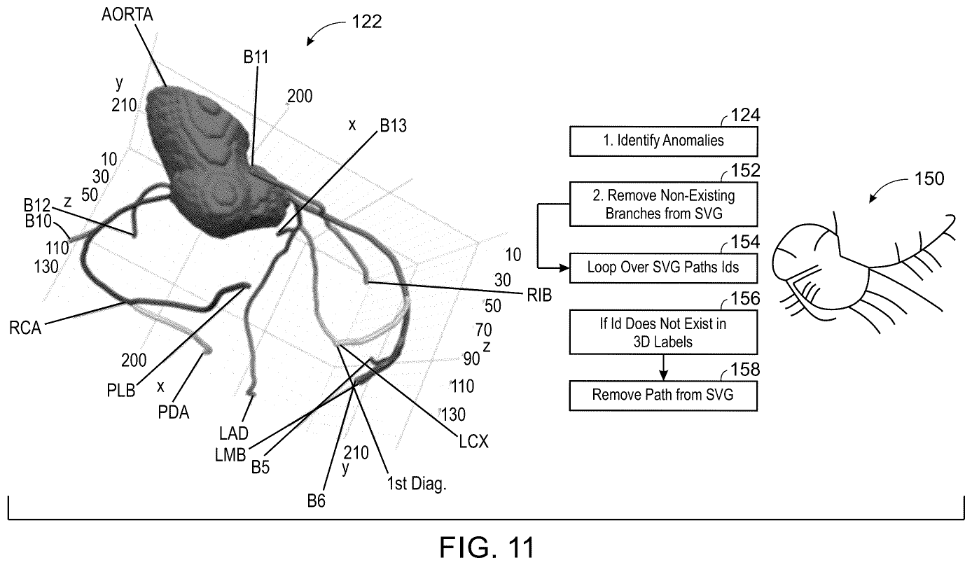

FIG. 11 depicts a schematic diagram of a second step in manipulating a standardized 2D coronary base schematic representation of a generic coronary tree. The left side of FIG. 11 depicts the image 122 of the 3D coronary tree of the subject (derived from cardiac CT scan data) utilized to manipulate a standardized 2D coronary base schematic representation of a generic coronary tree. The right side of FIG. 11 depicts the standardized 2D coronary base schematic representation 150 of the generic coronary tree after the initial manipulation (i.e., identification of any anomalies in the 3D coronary tree 122 of the subject as described in FIG. 8) or a first intermediate 2D coronary personalized schematic representation of the coronary tree of the subject. The second step includes removing any non-existing branches from the standardized 2D coronary base schematic representation 150 (i.e., SVG) that does not exist in the 3D coronary tree 122 as indicated by reference numeral 152. For example, the SVG path ids (i.e., each path element) are looped over as indicated by reference numeral 154. If the id does not exist in 3D labels of the 3D coronary tree 122 as indicated by reference numeral 156, the path is removed from the standardized 2D coronary base schematic representation 150 (i.e., SVG) as indicated by reference numeral 158.

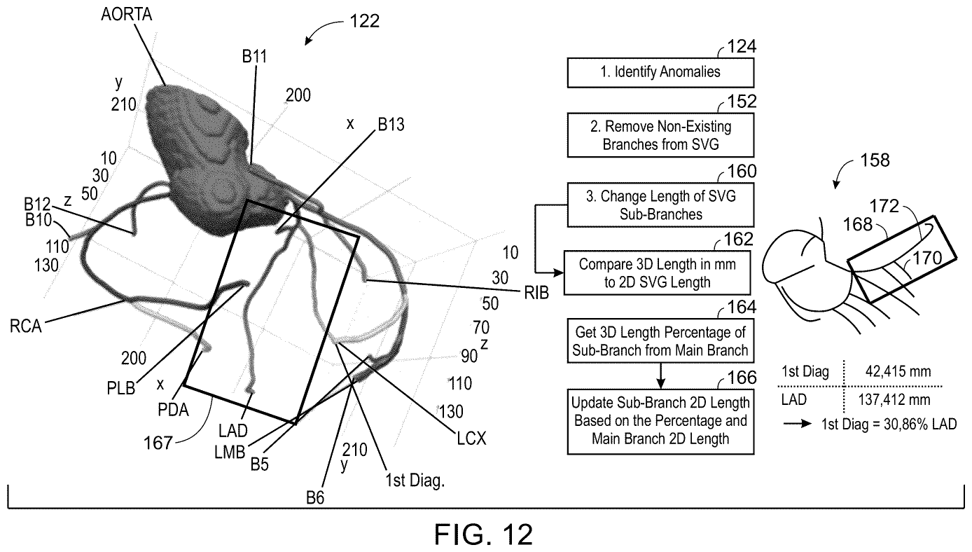

FIG. 12 depicts a schematic diagram of a third step in manipulating a standardized 2D coronary base schematic representation of a generic coronary tree. The left side of FIG. 12 depicts the image 122 of the 3D coronary tree of the subject (derived from cardiac CT scan data) utilized to manipulate a standardized 2D coronary base schematic representation of a generic coronary tree. The right side of FIG. 12 depicts the standardized 2D coronary base schematic representation 158 of the generic coronary tree after removal of non-existent branches in FIG. 11 or a second intermediate 2D coronary personalized schematic representation of the coronary tree of the subject. As depicted, the standardized 2D coronary base schematic representation 158 has had some branches removed when compared to the standardized 2D coronary base schematic representation 150. The third step includes changing the respective length of sub-branches in the standardized 2D coronary base schematic representation 158 as indicated by reference numeral 160. For example, a 3d length in millimeters of each respective sub-branch in the 3D coronary tree 122 is compared to the respective 2D length of the respective sub-branch in the standardized 2D coronary base schematic representation 158 (i.e., SVG) as indicated by reference numeral 162. This comparison includes getting the 3D length percentage of the respective sub-branch relative to the main branch in the 3D coronary tree 122 as indicated by reference numeral 164. Then, the corresponding sub-branch 2D length in the standardized 2D coronary base schematic representation 158 is updated based on the percentage and main branch 2D length in the standardized 2D coronary base schematic representation 158 as indicated by reference numeral 166. For example, rectangle 167 on the 3D coronary tree 122 and rectangle 168 on the standardized 2D coronary base schematic representation 158 highlights a sub-branch 170 (1st Diag) for which this adjustment in length is being carried out. In particular, this sub-branch 170 is being updated based on its percentage relative to LAD 172 and the length of LAD 172.

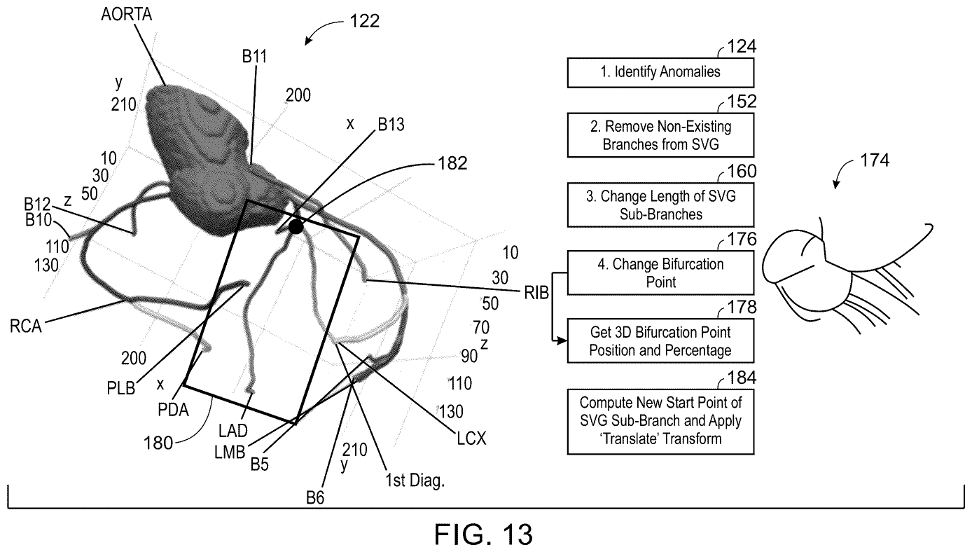

FIG. 13 depicts a schematic diagram of a fourth step in manipulating a standardized 2D coronary base schematic representation of a generic coronary tree. The left side of FIG. 13 depicts the image 122 of the 3D coronary tree of the subject (derived from cardiac CT scan data) utilized to manipulate a standardized 2D coronary base schematic representation of a generic coronary tree. The right side of FIG. 13 depicts the standardized 2D coronary base schematic representation 174 of the generic coronary tree after changing lengths of sub-branches in FIG. 12 or a third intermediate 2D coronary personalized schematic representation of the coronary tree of the subject. As depicted, the standardized 2D coronary base schematic representation 174 has had some branches removed when compared to the standardized 2D coronary base schematic representation 150. The fourth step includes changing a bifurcation point as indicated by reference numeral 176. This includes getting a 3D bifurcation point in the 3D coronary tree 122 as indicated by reference numeral 178. With rectangle 180 on the 3D coronary tree 122 is a bifurcation point 182 being analyzed. Upon getting the 3D bifurcation point, a new start position of a sub-branch in the standardized 2D coronary base schematic representation 174 (i.e., SVG) is computed and translation transformation is applied as indicated by reference numeral 184.

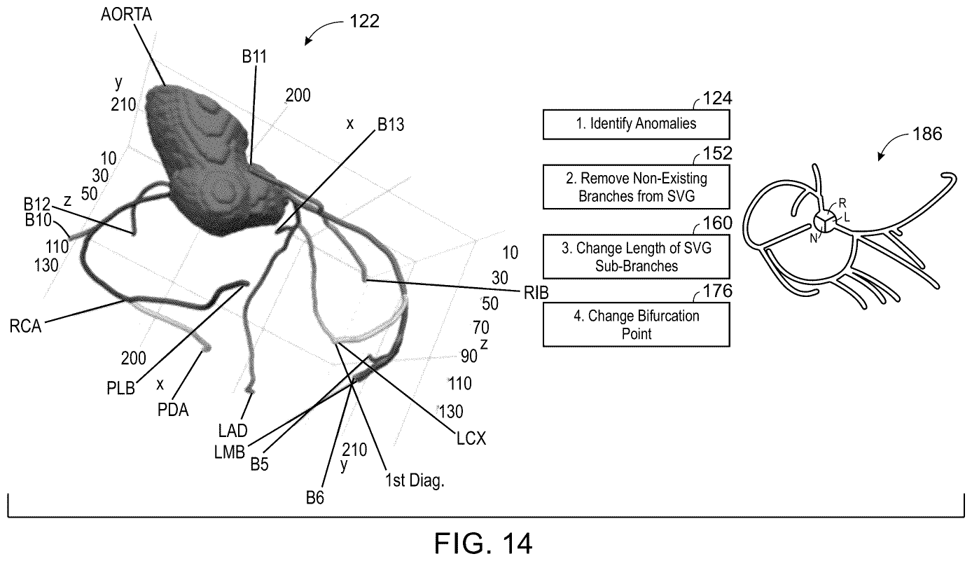

FIG. 14 depicts a schematic diagram of the generation of a standardized 2D coronary personalized representation of a coronary tree of a subject. The left side of FIG. 14 depicts the image 122 of the 3D coronary tree of the subject (derived from cardiac CT scan data) utilized to manipulate a standardized 2D coronary base schematic representation of a generic coronary tree. The middle of FIG. 14 depicts the steps utilized to modify a standardized 2D coronary base schematic representation of a generic coronary tree to generate a standardized 2D coronary personalized representation 186 of a coronary tree of a subject based on the 3D coronary tree 122 of the subject. The right side of FIG. 14 depicts the standardized 2D coronary personalized representation 186 of the coronary tree of the subject after changing the bifurcation point in FIG. 13.

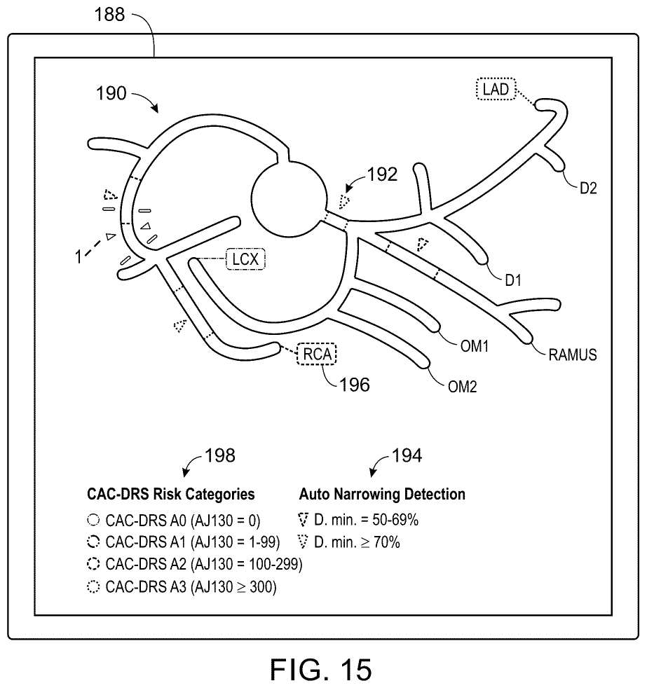

FIG. 15 depicts a user interface 188 depicting a final rendering of a standardized 2D coronary personalized representation of a coronary tree of a subject. The user interface 188 may be displayed on the display device 50 in FIG. 2. The user interface 188 depicts a standardized 2D coronary personalized representation 190 of a coronary tree of a subject generated as described above. The standardized 2D coronary personalized representation 190 includes additional information derived from the analysis of the cardiac CT scan data of the subject from which the standardized 2D coronary personalized representation 190 is derived. For examples, areas of narrowing are indicated by arrows 192 on the standardized 2D coronary personalized representation 190. The arrows 192 may be color coded to indicate the severity of narrowing. The user interface 188 depicts a legend 194 for the levels of narrowing indicated by the arrows 192. Labels 196 identifying the various branches of the standardized 2D coronary personalized representation 190 are color coded to indicate a calcification score for the respective branches utilizing a standardized reporting method (e.g., Coronary Artery Calcium Data and Reporting System (CAC-DRS)). The user interface 188 depicts a legend 198 for the levels of calcification as indicated by the color coding of the labels. The presentation of the additional information relative to the standardized 2D coronary personalized representation 190 may from that depicted in FIG. 15.

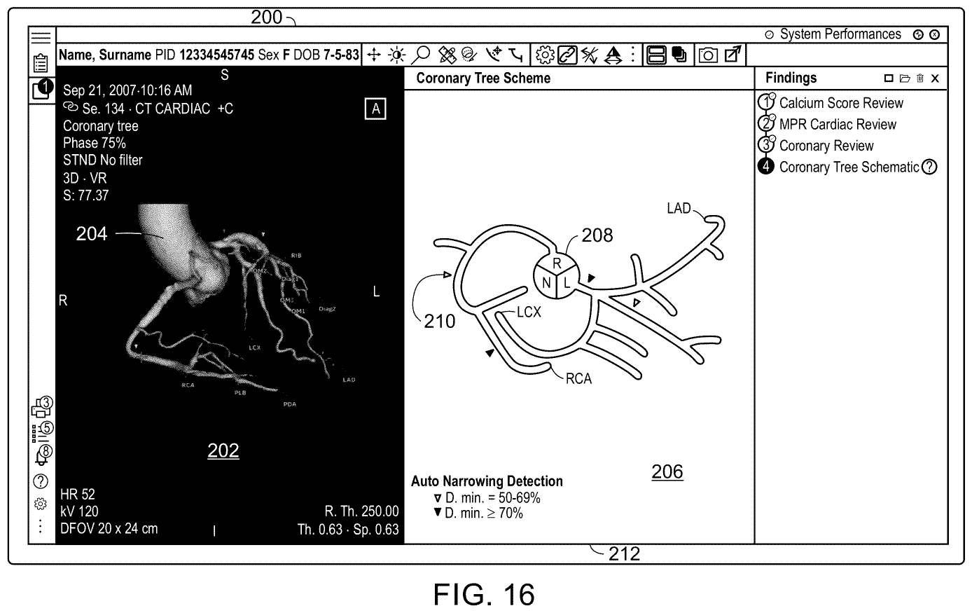

FIG. 16 depicts another user interface 200 depicting a 3D coronary tree of a subject and a final rendering of a standardized 2D coronary personalized representation of a coronary tree of the subject derived from the 3D coronary tree. The user interface 200 may be displayed on the display device 50 in FIG. 2. A left panel 202 of the user interface 200 depicts a labeled 3D coronary tree 204 segmented from cardiac CT scan data of a subject. A right panel 206 of the user interface 188 depicts a standardized 2D coronary personalized representation 208 of a coronary tree of a subject generated as described above based on the 3D coronary tree 204. The standardized 2D coronary personalized representation 208 includes additional information derived from the analysis of the cardiac CT scan data of the subject from which the standardized 2D coronary personalized representation 208 is derived. For examples, areas of narrowing are indicated by arrows 210 on the standardized 2D coronary personalized representation 208. The arrows 210 may be color coded to indicate the severity of narrowing. The user interface 200 depicts a legend 212 for the levels of narrowing indicated by the arrows 210.

Technical effects of the disclosed embodiments include generating customized schematic views of coronary arteries (e.g., display on a graphical user interface). In particular, technical effects of the disclosed embodiments include generating of a simplified, personalized 2D diagram of segmented cardiovascular systems based on a standard (e.g., provided by SCCT). Technical effects of the disclosed embodiments include providing a generalized or base 2D schematic representation of the coronary arteries that is adapted to integrate personalized features from automatic 3D coronary segmentation and labeling to generate a personalized schematic representation of a coronary tree of the patient (e.g., subject). Technical effects of the disclosed embodiments include providing a more accessible and comprehensive visualization tool to improve communication among healthcare professionals and enhance the overall quality of coronary artery disease assessment and treatment planning.

The disclosure also provides support for a computer-implemented method for generating a customized schematic view of coronary arteries, comprising: obtaining, via a processing system comprising one or more processors, a three-dimensional (3D) coronary tree segmented from computed tomography (CT) cardiac scan data of a subject, wherein the 3D coronary tree is labeled; obtaining, via the processing system, a standardized two-dimensional (2D) coronary base schematic representation of a generic coronary tree; and modifying, via the processing system, the standardized 2D coronary base schematic representation of the generic coronary tree based on the 3D coronary tree to generate a standardized 2D coronary personalized schematic representation of a coronary tree of the subject. In a first example of the computer-implemented method, the computer-implemented method further comprises displaying, via the processing system, the standardized 2D coronary personalized schematic representation of the coronary tree of the subject on a graphical user interface. In a second example of the computer-implemented method, optionally including the first example, an appearance of the standardized 2D coronary personalized schematic representation of the coronary tree of the subject on the graphical user interface is independent of viewing angle. In a third example of the computer-implemented method, optionally including one or both of the first and second examples, the standardized 2D coronary base schematic representation of the generic coronary tree is standardized based on standardized coronary segmentation tree diagram provided by the Society of Cardiovascular Computed Tomography (SCCT). In a fourth example of the computer-implemented method, optionally including one or more or each of the first through third examples, the standardized 2D coronary base schematic representation of the generic coronary tree is derived from a combination of a right-dominant SCCT coronary tree segmentation diagram, a co-dominant SCCT coronary segmentation tree diagram, and a left-dominant SCCT coronary tree segmentation diagram. In a fifth example of the computer-implemented method, optionally including one or more or each of the first through fourth examples, the computer-implemented method further comprises, prior to modifying the standardized 2D coronary base schematic representation of the generic coronary tree, identifying, via the processing system, unknown branches in the 3D coronary tree. In a sixth example of the computer-implemented method, optionally including one or more or each of the first through fifth examples, identifying unknown branches in the 3D coronary tree comprises: defining, via the processing system, a list of unknown branches with their coordinates; defining, via the processing system, a list of main branches with their coordinates; defining, via the processing system, a list of possible sub-branches for the main branches from the list of unknown branches; identifying, via the processing system, existing sub-branches of each main branch; identifying, via the processing system, common parts between a respective unknown branch from the list of possible sub-branches and a respective main branch; determining, via the processing system, that a respective common part is a sub-branch when the respective common part is greater than an aorta of the 3D coronary tree; and adding, via the processing system, the determined sub-branch to the identified existing sub-branches. In an seventh example of the computer-implemented method, optionally including one or more or each of the first through sixth examples, modifying the standardized 2D coronary base schematic representation of the generic coronary tree based on the 3D coronary tree comprises: identifying, via the processing system, anomalies in the 3D coronary tree; removing, via the processing system, any branch from the standardized 2D coronary base schematic representation of the generic coronary tree that does not exist in the 3D coronary tree; changing, via the processing system, a respective length of one or more sub-branches of the standardized 2D coronary base schematic representation of the generic coronary tree based on a corresponding respective length of the one or more corresponding sub-branches in the 3D coronary tree; and changing, via the processing system, a bifurcation point of the standardized 2D coronary base schematic representation of the generic coronary tree based on a corresponding bifurcation point of the 3D coronary tree.

The disclosure also provides support for a system for generating a customized schematic view of coronary arteries, comprising: a memory encoding processor-executable routines; and a processor comprising one or more processors and configured to access the memory and to execute the processor-executable routines, wherein the processor-executable routines, when executed by the processing system, cause the processing system to: obtain a three-dimensional (3D) coronary tree segmented from computed tomography (CT) cardiac scan data of a subject, wherein the 3D coronary tree is labeled; obtain a standardized two-dimensional (2D) coronary base schematic representation of a generic coronary tree; and modify the standardized 2D coronary base schematic representation of the generic coronary tree based on the 3D coronary tree to generate a standardized 2D coronary personalized schematic representation of a coronary tree of the subject. In a first example of a system, the processor-executable routines, when executed by the processing system, further cause the processing system to display the standardized 2D coronary personalized schematic representation of the coronary tree of the subject on a graphical user interface. In a second example of the system, optionally including the first example, an appearance of the standardized 2D coronary personalized schematic representation of the coronary tree of the subject on the graphical user interface is independent of viewing angle. In a third example of the system, optionally including one or both of the first and second examples, the standardized 2D coronary base schematic representation of the generic coronary tree is standardized based on standardized coronary segmentation tree diagram provided by the Society of Cardiovascular Computed Tomography (SCCT). In a fourth example of the system, optionally including one or more or each of the first through third examples, the standardized 2D coronary base schematic representation of the generic coronary tree is derived from a combination of a right-dominant SCCT coronary tree segmentation diagram, a co-dominant SCCT coronary segmentation tree diagram, and a left-dominant SCCT coronary tree segmentation diagram. In a fifth example of the system, optionally including one or more or each of the first through fourth examples, the processor-executable routines, when executed by the processing system, further cause the processing system, prior to modifying the standardized 2D coronary base schematic representation of the generic coronary tree, to identify unknown branches in the 3D coronary tree. In a sixth example of the system, optionally including one or more or each of the first through fifth examples, identifying unknown branches in the 3D coronary tree comprises: defining a list of unknown branches with their coordinates; defining a list of main branches with their coordinates; defining a list of possible sub-branches for the main branches from the list of unknown branches; identifying existing sub-branches of each main branch; identifying common parts between a respective unknown branch from the list of possible sub-branches and a respective main branch; determining that a respective common part is a sub-branch when the respective common part is greater than an aorta of the 3D coronary tree; and adding the determined sub-branch to the identified existing sub-branches. In a seventh example of the system, optionally including one or more or each of the first through sixth examples, modifying the standardized 2D coronary base schematic representation of the generic coronary tree based on the 3D coronary tree comprises: identifying, via the processing system, anomalies in the 3D coronary tree; removing, via the processing system, any branch from the standardized 2D coronary base schematic representation of the generic coronary tree that does not exist in the 3D coronary tree; changing, via the processing system, a respective length of one or more sub-branches of the standardized 2D coronary base schematic representation of the generic coronary tree based on a corresponding respective length of the one or more corresponding sub-branches in the 3D coronary tree; and changing, via the processing system, a bifurcation point of the standardized 2D coronary base schematic representation of the generic coronary tree based on a corresponding bifurcation point of the 3D coronary tree.

The disclosure also provides support for a non-transitory computer-readable medium, the computer-readable medium comprising processor-executable code that when executed by a processing system comprising one or more processors, causes the processing system to: obtain a three-dimensional (3D) coronary tree segmented from computed tomography (CT) cardiac scan data of a subject, wherein the 3D coronary tree is labeled; obtain a standardized two-dimensional (2D) coronary base schematic representation of a generic coronary tree; and modify the standardized 2D coronary base schematic representation of the generic coronary tree based on the 3D coronary tree to generate a standardized 2D coronary personalized schematic representation of a coronary tree of the subject. In a first example of the non-transitory computer-readable medium, the processor-executable code, when executed by the processing system, further causes the processing system to display the standardized 2D coronary personalized schematic representation of the coronary tree of the subject on a graphical user interface. In a second example of the non-transitory computer-readable medium, optionally including the first example, an appearance of the standardized 2D coronary personalized schematic representation of the coronary tree of the subject on the graphical user interface is independent of viewing angle. In a third example of the non-transitory computer-readable medium, optionally including one or both of the first and second examples, wherein modifying the standardized 2D coronary base schematic representation of the generic coronary tree based on the 3D coronary tree comprises: identifying anomalies in the 3D coronary tree; removing any branch from the standardized 2D coronary base schematic representation of the generic coronary tree that does not exist in the 3D coronary tree; changing a respective length of one or more sub-branches of the standardized 2D coronary base schematic representation of the generic coronary tree based on a corresponding respective length of the one or more corresponding sub-branches in the 3D coronary tree; and changing a bifurcation point of the standardized 2D coronary base schematic representation of the generic coronary tree based on a corresponding bifurcation point of the 3D coronary tree.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform]ing [a function] . . . ”, it is intended that such elements are to be interpreted under 35 U.S.C. 112(f). However, for any claims containing elements designated in any other manner, it is intended that such elements are not to be interpreted under 35 U.S.C. 112(f).

This written description uses examples to disclose the present subject matter, including the best mode, and also to enable any person skilled in the art to practice the subject matter, including making and using any devices or systems and performing any incorporated methods. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A computer-implemented method for generating a customized schematic view of coronary arteries, comprising:
obtaining, via a processing system comprising one or more processors, a three-dimensional (3D) coronary tree segmented from computed tomography (CT) cardiac scan data of a subject, wherein the 3D coronary tree is labeled;
obtaining, via the processing system, a standardized two-dimensional (2D) coronary base schematic representation of a generic coronary tree; and
modifying, via the processing system, the standardized 2D coronary base schematic representation of the generic coronary tree based on the 3D coronary tree to generate a standardized 2D coronary personalized schematic representation of a coronary tree of the subject.

2. The computer-implemented method of claim 1, further comprising displaying, via the processing system, the standardized 2D coronary personalized schematic representation of the coronary tree of the subject on a graphical user interface.

3. The computer-implemented method of claim 2, wherein an appearance of the standardized 2D coronary personalized schematic representation of the coronary tree of the subject on the graphical user interface is independent of viewing angle.

4. The computer-implemented method of claim 1, wherein the standardized 2D coronary base schematic representation of the generic coronary tree is standardized based on a standardized coronary segmentation tree diagram provided by the Society of Cardiovascular Computed Tomography (SCCT).

5. The computer-implemented method of claim 4, wherein the standardized 2D coronary base schematic representation of the generic coronary tree is derived from a combination of a right-dominant SCCT coronary tree segmentation diagram, a co-dominant SCCT coronary segmentation tree diagram, and a left-dominant SCCT coronary tree segmentation diagram.

6. The computer-implemented method of claim 1, further comprising, prior to modifying the standardized 2D coronary base schematic representation of the generic coronary tree, identifying, via the processing system, unknown branches in the 3D coronary tree.

7. The computer-implemented method of claim 6, wherein identifying unknown branches in the 3D coronary tree comprises:
defining, via the processing system, a list of unknown branches with their coordinates;
defining, via the processing system, a list of main branches with their coordinates;
defining, via the processing system, a list of possible sub-branches for the main branches from the list of unknown branches;
identifying, via the processing system, existing sub-branches of each main branch;
identifying, via the processing system, common parts between a respective unknown branch from the list of possible sub-branches and a respective main branch;
determining, via the processing system, that a respective common part is a sub-branch when the respective common part is greater than an aorta of the 3D coronary tree; and
adding, via the processing system, the determined sub-branch to the identified existing sub-branches.

8. The computer-implemented method of claim 1, wherein modifying the standardized 2D coronary base schematic representation of the generic coronary tree based on the 3D coronary tree comprises:
identifying, via the processing system, anomalies in the 3D coronary tree;
removing, via the processing system, any branch from the standardized 2D coronary base schematic representation of the generic coronary tree that does not exist in the 3D coronary tree;
changing, via the processing system, a respective length of one or more sub-branches of the standardized 2D coronary base schematic representation of the generic coronary tree based on a corresponding respective length of the one or more corresponding sub-branches in the 3D coronary tree; and
changing, via the processing system, a bifurcation point of the standardized 2D coronary base schematic representation of the generic coronary tree based on a corresponding bifurcation point of the 3D coronary tree.

9. A system for generating a customized schematic view of coronary arteries, comprising:
a memory encoding processor-executable routines; and
a processor comprising one or more processors and configured to access the memory and to execute the processor-executable routines, wherein the processor-executable routines, when executed by the processing system, cause the processing system to:
obtain a three-dimensional (3D) coronary tree segmented from computed tomography (CT) cardiac scan data of a subject, wherein the 3D coronary tree is labeled;
obtain a standardized two-dimensional (2D) coronary base schematic representation of a generic coronary tree; and
modify the standardized 2D coronary base schematic representation of the generic coronary tree based on the 3D coronary tree to generate a standardized 2D coronary personalized schematic representation of a coronary tree of the subject.

10. The system of claim 9, wherein the processor-executable routines, when executed by the processing system, further cause the processing system to display the standardized 2D coronary personalized schematic representation of the coronary tree of the subject on a graphical user interface.

11. The system of claim 10, wherein an appearance of the standardized 2D coronary personalized schematic representation of the coronary tree of the subject on the graphical user interface is independent of viewing angle.

12. The system of claim 9, wherein the standardized 2D coronary base schematic representation of the generic coronary tree is standardized based on a standardized coronary segmentation tree diagram provided by the Society of Cardiovascular Computed Tomography (SCCT).

13. The system of claim 12, wherein the standardized 2D coronary base schematic representation of the generic coronary tree is derived from a combination of a right-dominant SCCT coronary tree segmentation diagram, a co-dominant SCCT coronary segmentation tree diagram, and a left-dominant SCCT coronary tree segmentation diagram.

14. The system of claim 9, wherein the processor-executable routines, when executed by the processing system, further cause the processing system, prior to modifying the standardized 2D coronary base schematic representation of the generic coronary tree, to identify unknown branches in the 3D coronary tree.

15. The system of claim 14, wherein identifying unknown branches in the 3D coronary tree comprises:
defining a list of unknown branches with their coordinates;
defining a list of main branches with their coordinates;
defining a list of possible sub-branches for the main branches from the list of unknown branches;
identifying existing sub-branches of each main branch;
identifying common parts between a respective unknown branch from the list of possible sub-branches and a respective main branch;
determining that a respective common part is a sub-branch when the respective common part is greater than an aorta of the 3D coronary tree; and
adding the determined sub-branch to the identified existing sub-branches.

16. The system of claim 9, wherein modifying the standardized 2D coronary base schematic representation of the generic coronary tree based on the 3D coronary tree comprises:
identifying anomalies in the 3D coronary tree;
removing any branch from the standardized 2D coronary base schematic representation of the generic coronary tree that does not exist in the 3D coronary tree;
changing a respective length of one or more sub-branches of the standardized 2D coronary base schematic representation of the generic coronary tree based on a corresponding respective length of the one or more corresponding sub-branches in the 3D coronary tree; and
changing a bifurcation point of the standardized 2D coronary base schematic representation of the generic coronary tree based on a corresponding bifurcation point of the 3D coronary tree.

17. A non-transitory computer-readable medium, the computer-readable medium comprising processor-executable code that when executed by a processing system comprising one or more processors, causes the processing system to:
obtain a three-dimensional (3D) coronary tree segmented from computed tomography (CT) cardiac scan data of a subject, wherein the 3D coronary tree is labeled;
obtain a standardized two-dimensional (2D) coronary base schematic representation of a generic coronary tree; and
modify the standardized 2D coronary base schematic representation of the generic coronary tree based on the 3D coronary tree to generate a standardized 2D coronary personalized schematic representation of a coronary tree of the subject.

18. The non-transitory computer-readable medium of claim 17, wherein the processor-executable code, when executed by the processing system, further causes the processing system to display the standardized 2D coronary personalized schematic representation of the coronary tree of the subject on a graphical user interface.

19. The non-transitory computer-readable medium of claim 18, wherein an appearance of the standardized 2D coronary personalized schematic representation of the coronary tree of the subject on the graphical user interface is independent of viewing angle.

20. The non-transitory computer-readable medium of claim 17, wherein modifying the standardized 2D coronary base schematic representation of the generic coronary tree based on the 3D coronary tree comprises:
identifying anomalies in the 3D coronary tree;
removing any branch from the standardized 2D coronary base schematic representation of the generic coronary tree that does not exist in the 3D coronary tree;
changing a respective length of one or more sub-branches of the standardized 2D coronary base schematic representation of the generic coronary tree based on a corresponding respective length of the one or more corresponding sub-branches in the 3D coronary tree; and
changing a bifurcation point of the standardized 2D coronary base schematic representation of the generic coronary tree based on a corresponding bifurcation point of the 3D coronary tree.
